(19) United States
(12) Patent Application Publication
(10) Pub. No.: US 2026/0174648 A1
(43) Pub. Date: Jun. 25, 2026

(54) ZERO-WASTE ECO-FRIENDLY COSMETIC PRODUCT AND METHOD FOR MANUFACTURING SAME

(71) Applicant: COSMAX, INC., Gyeonggi-do (KR)

(72) Inventors: Hyun Ju PARK, Gyeonggi-do (KR); Eun Ju CHOI, Gyeonggi-do (KR); Eun Ju SEO, Gyeonggi-do (KR)

(21) Appl. No.: 19/124,458

(22) PCT Filed: Oct. 19, 2023
(86) PCT No.: PCT/KR2023/016246

§ 371 Date: Apr. 25, 2025

(30) Foreign Application Priority Data
Oct. 27, 2022 (KR) 10-2022-0140742

(51) Int. Cl. A61K 8/365; A61K 8/31; A61K 8/37; A61K 8/58; A61Q 1/02

(52) U.S. Cl. CPC A61K 8/365; A61K 8/31; A61K 8/37; A61K 8/585; A61Q 1/02

(57) ABSTRACT

The present invention relates to a zero-waste eco-friendly cosmetic product and a method for manufacturing same. The present invention provides a cosmetic product that does not contain plastic and minimizes the generation of waste and thus is eco-friendly and can be used by cutting. In addition, the method for manufacturing a cosmetic product according to the present invention has the effect of ensuring stability by minimizing pinholes and molding marks which can be formed in the cosmetic content.

### TECHNICAL FIELD

The present disclosure relates to a zero-waste eco-friendly cosmetic product and a method for manufacturing the same and, more specifically, to a cosmetic product, which uses a raw material applicable thereto in place of plastics in a cosmetic case and enables the reuse of the case left after the use of the cosmetic product.

### BACKGROUND ART

Plastics used for cosmetic cases make 60,000 tons of plastic waste annually, of which more than 90% is non-recyclable trash. In other words, the plastics enter the environment through incineration or landfilling, causing harm to the Earth environment. Thus, efforts are needed to manufacture cosmetic products requiring no plastic cases, manufacture cosmetic cases by using materials that can replace plastics used for cosmetic cases, for example, raw materials applicable to cosmetic products, or reduce waste by reusing cases left after the use of cosmetic products.

For instance, cosmetic pencils are stick-type makeup tools that are composed of a core corresponding to the cosmetic content and a body corresponding to a cosmetic case and can be sharpened like a pencil for use. Cosmetic pencils, like eyeshadows, eyebrows, eyeliners, lips, lip liners, or concealers, can be offered with various cosmetic materials as cores, such as eyeshadows, eyebrows, eyeliners, lips, lip liners, or concealers.

Bodies (outer cases) of typical cosmetic pencils are formed of a wood or plastic material. Cosmetic pencils that use a wooden body, like pencils, generate dust and employ plasticizers or adhesives during the processing of raw materials. Moreover, this may cause harm to the natural environment, such as hastening desertification and disrupting ecosystems due to deforestation.

Consequently, there is a need to develop a cosmetic product and a manufacturing method therefor, wherein the cosmetic product contains no plastics, is eco-friendly, and is designed to be sharpened for use.

### DISCLOSURE

### Technical Problem

The present disclosure has been made to solve the above-described issues, and an aspect of the present disclosure is to provide a cosmetic product that uses a raw material applicable thereto but not contains a plastic material in a case or body.

Another aspect of the present disclosure is to provide an eco-friendly cosmetic product, which includes a case (a body) and a cosmetic content (core) inside the case integrated into a single unit, can be sharpened with a sharpener for use, and keeps the cosmetic content from getting on a user's hands.

Yet another aspect of the present disclosure is to provide an eco-friendly cosmetic product capable of expressing a gorgeous outline by applying colors and laser engraving to the body of the cosmetic product.

Still yet another aspect of the present disclosure is to provide an eco-friendly cosmetic product capable of utilizing, as a cube candle, a case body left after the use of the cosmetic product.

Still yet another aspect of the present disclosure is to provide a method for molding an eco-friendly cosmetic product.

### Technical Solution

An aspect of the present disclosure provides a cosmetic product including a body and a core.

The body may have a predetermined width and a predetermined length, wherein the body is elongated in the longitudinal direction and may have a length of 30 to 200 mm.

The core may be elongated in the longitudinal direction of the body, inside the body, and may contain a cosmetic composition, wherein the core may have a width of 2 to 20 mm.

The body may be formed of a wax material, and may further contain an oil, and may further contain at least one selected from the group consisting of a colorant, pearl, and a fragrance material.

The cosmetic product may be in a pencil form.

The cosmetic product may include a lid coupled to either end of the body, and the lid may contain at least one material selected from the group consisting of wax, oil, cellulose, gelatin, paper, felt paper, and leather.

In accordance with another aspect of the present disclosure, a method for manufacturing a cosmetic product is provided including a core molding step and a body molding step.

In the core molding step, a core composition is filled into a first mold, followed by cooling and releasing, thereby preparing a core molded body.

In the body molding step, a body composition is filled into the area outside the core molded body while the core body is inserted into a second mold, followed by cooling and releasing, thereby molding a body.

In the body molding step, the body composition is filled to be equal to or higher than the height of the core molded body.

### Advantageous Effects

The cosmetic product according to the present disclosure contains no plastic materials and uses a raw material applicable thereto and thus is environmentally preferable.

Furthermore, the cosmetic product according to the present disclosure, which includes a body and a core integrated into a single unit, can be sharpened with a sharpener for use and can keep the cosmetic content from getting on the hands.

Furthermore, the cosmetic product according to the present disclosure can express a gorgeous outline by applying colors and laser engraving to the outer circumferential surface of the body of the cosmetic product.

Furthermore, the body left after the use of the cosmetic product can be used as a cube candle, thereby minimizing the generation of waste.

Furthermore, in the method for manufacturing a cosmetic product according to the present disclosure, a double filling process of primarily filling the core and secondarily filling the body can be employed to minimize pinholes that may be caused in the core containing a cosmetic composition, thereby ensuring stability of the cosmetic product.

### BRIEF DESCRIPTION OF DRAWINGS

FIG. 1A schematically shows a cross section of a cosmetic pencil as an example of a cosmetic product of the present disclosure.

FIG. 1B illustrates the use of a cosmetic pencil manufactured according to an embodiment of the present disclosure.

FIG. 2 is a flow chart schematically showing a method for manufacturing a cosmetic pencil according to an embodiment of the present disclosure.

FIGS. 3A and 3B schematically show molds used in respective steps in the manufacturing method of the present disclosure.

### BEST MODE FOR INVENTION

Hereinafter, the present disclosure will be described in detail with reference to exemplary embodiments. However, these exemplary embodiments are for illustrative purposes only, and the scope of the present disclosure is not intended to be limited thereto.

An aspect of the present disclosure provides an eco-friendly cosmetic product.

The cosmetic product may include: a body with a predetermined width and a predetermined length, the body being elongated in the longitudinal direction; and a core elongated in the longitudinal direction of the body, inside the body. The cosmetic product may further include a lid coupled to either end of the body.

The body may be formed of a wax material, and the core may contain a cosmetic composition.

The lid may contain at least one material selected from the group consisting of wax, oil, cellulose, gelatin, paper, felt paper, and leather.

The body may have a length of 30 to 200 mm, and the core may have a width of 2 to 20 mm.

The body is left after the use of the core and can be utilized as a cube candle. The cube candle refers to a cube-shaped candle that is placed in a candle warmer for a cube candle. The candle warmer is a tool that generates heat from a light bulb to melt a scented candle, thereby releasing its scent. The candle warmer for a cube candle is a tool that applies heat to a downwardly concave bowl, where a cube-shaped candle is placed, from a light bulb to melt the candle inside the bowl, thereby releasing its scent.

According to the present disclosure, the body left after the use of the core corresponding to the cosmetic content is utilized as a cube candle, thereby minimizing waste, that is, the generation of cases left after the use of the cosmetic composition.

Another aspect of the present disclosure provides a method for manufacturing an eco-friendly cosmetic product.

The method may include: a core molding step of filling a core composition into a first mold, followed by cooling and releasing, thereby preparing a core molded body; and a body molding step of filling a body composition into the area outside the core molded body while the core body is inserted into a second mold, followed by cooling and releasing, thereby molding a body. In the body molding step, the body composition may be filled to be equal to or higher than the height of the core molded body.

Hereinafter, the present disclosure will be described in more detail with reference to drawings.

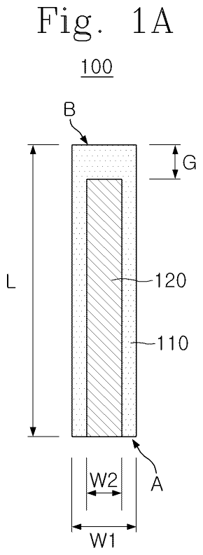

FIG. 1A is a cross-sectional diagram schematically showing a cosmetic pencil 100 as one example of the cosmetic product of the present disclosure, and FIG. 1B illustrates an example of the use of a cosmetic pencil 100 manufactured according to an embodiment of the present disclosure and shows that a core is protruded by processing of an end in FIG. 1A.

Referring to FIGS. 1A and 1B, the pencil 100 includes a body 110 and a core 120.

As shown, the body 110 is a column-shaped molded body that has a predetermined width (W1) and length (L) and is elongated in the longitudinal direction.

For instance, the body 110 is shown to have a column shape, but the shape of the body of the present disclosure is not limited thereto. For example, the body 110 may have a polygonal column shape, such as a rectangular column or a hexagonal column.

In the present disclosure, the length of the body may be 30 to 200 mm. In a preferable embodiment, the length of the body is preferably 30 mm or more, 50 mm or more, or 70 mm or more, and preferably 200 mm or less, 150 mm or less, or 100 mm or less. If the length of the body is less than 30 mm, the pencil is short, making it difficult to hold and use or sharpen with a sharpener for use, and if the length of the body is more than 200 mm, the pencil may be easily broken.

Additionally, the core 120 may be a molded boy of a cosmetic composition that has a width (W2) smaller than the width (W1) of the body 110 and is elongated in the longitudinal direction of the body 110, inside the body 110. The core 120 may be a molded body with a column shape, like the body 110.

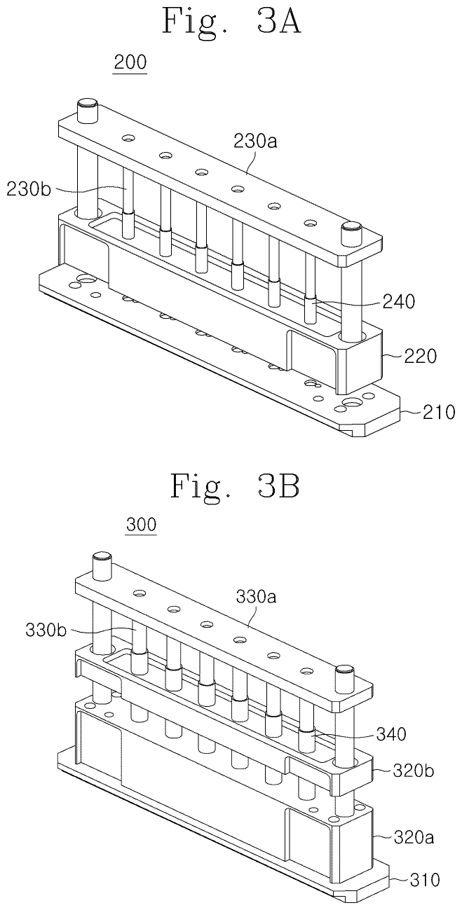

As shown, the core 120 may be exposed through one end (A) of both ends of the body 110, while the core 120 may not be exposed through the other end (B) of the pencil 100. A molding method for giving a height difference (G) not to expose the core 120 through the end (B) in FIG. 1 will be described later with reference to FIG. 3.

This configuration is for protecting the core and minimizing the occurrence of pinholes in a two-step filling manner, which corresponds to an exemplary manufacturing method of the present disclosure. The present disclosure is not limited thereto, and the pencil of the present disclosure may be formed such that both ends of the core may be exposed or may not be exposed.

The width (W2) of the core may be 2 to 20 mm. In a preferable embodiment, the width (W2) of the core is preferably 2 mm or more, 5 mm or more, or 10 mm or more, and preferably 20 mm or less, 15 mm or less, or 12 mm or less. If the width (W2) of the core is less than 2 mm, the pencil may be broken during the sharpening of the core with a sharpener for use, and if the width (W2) of the core is more than 20 mm, the pencil is difficult to hold and use with one hand.

Additionally, the pencil 100 may further include a lid, which is coupled to one end of both the ends thereof. The lid may be coupled to one end or the other end of the pencil according to the state of use.

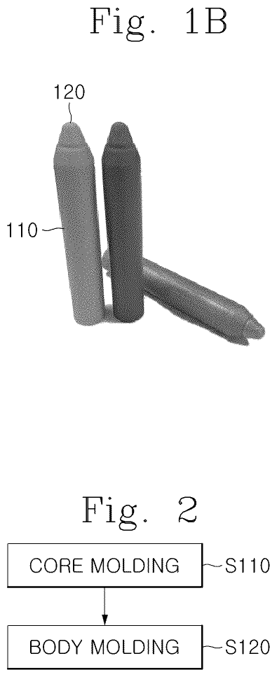

FIG. 2 is a flow chart showing an example of a method for manufacturing a cosmetic pencil according to an embodiment of the present disclosure.

Referring to FIG. 2, the pencil 100 may be manufactured in a two-step molding manner including a core molding step S110 and a body molding step S120.

Specifically, in the core molding step S110, a core is molded using a first mold. Subsequently, the body molding step S120 of molding a body outside the core molded body by using a second mold, so that a cosmetic pencil where the core is disposed inside the body may be manufactured.

FIGS. 3A and 3B schematically show the first mold used in the core molding step and the second mold used in the body molding step, respectively.

Referring to FIG. 3A, the core 120 may be prepared by the first mold 200.

Specifically, in the first mold 200, a first filling part 220 may be coupled onto a support part 210. The first mold 200 may include, for easy release, a fixing part 230 having a plate 230a to which columns 230b capable of fixing the molded cores are coupled.

In the core molding step S110, a core composition may be filled into the first filling part 220 coupled to the support part 210, followed by cooling and release, thereby manufacturing core molded bodies 240. The core molded bodies 240 may be fixed on the columns 230b of the fixing part 230 and may be released by lifting of the fixing part 230. In another embodiment, while the core molded bodies 240 are fixed on the support part 210, the first filling part 220 may be separated from the support part 210 and removed from the core molded bodies.

Referring to FIG. 3B, the body 110 may be prepared by the second mold 300 in the present disclosure.

Specifically, in the second mold 300, a second filling part 320 may be coupled onto a support part 310. The second mold 300 may include, for easy release, a fixing part 330 having a plate 330a to which columns 330b capable of fixing the molded bodies are coupled.

In the second filling part 320, a lower plate 320a and an upper plate 320b may be coupled to each other or may be incorporated into a single unit. Specifically, the height of the second filling part 320 is preferably equal to or higher than the height of the first filling part 220. The second filling part 320 is made to be higher than the core molded bodies and then filled with a body composition, so that the height difference (G) between the body and the core as shown in FIG. 1 may be formed.

In the body molding step S120, the second filling part is coupled to the support part 310, and then while the core molded bodies are inserted into the second filling part, the body composition may be used to fill the area outside the core molded bodies.

If the second filling part 320 consisting of the lower plate 320a and the upper plate 320b coupled to each other is used, a boundary line corresponding to a coupled portion of the lower plate 320a and the upper plate 320b is formed on the outer circumferential surface of the body, as shown in FIG. 1A, but if the second filling part 320 consisting of the lower plate 320a and the upper plate 320b incorporated into a single unit is used, the outer circumferential surface of the body has a smooth surface and exhibits excellent aesthetic expression, as shown in FIG. 1B.

The filled body composition is cooled and then released, thereby molding bodies 340. The core molded bodies 340 may be fixed on the columns 330b of the fixing part 330 and may be released by lifting of the fixing part 330. In another embodiment, while the body molded bodies 340 are fixed on the support part 310, the second filling part 320 may be separated from the support part 310 and removed from the core molded bodies.

### MODE FOR INVENTION

Hereinafter, the body composition and the core composition used in the two-step molding manner of the present disclosure will be described.

### A. Body Composition

The body composition may contain wax.

The wax may be an oily ingredient that is in a solid phase at room temperature (that is, 25° C.), and any known wax that is acceptable as a cosmetic ingredient may be used. For example, the wax may include at least one selected from the group consisting of shea butter, cacao butter, beeswax, paraffin wax, microcrystalline wax, candelilla wax, carnauba wax, synthetic wax, hydrogenated castor oil, and a mixture thereof, but is not limited thereto.

The body composition preferably contains, as an oily ingredient that is in a solid phase, at least one selected from the group consisting of hydrogenated soybean oil, microcrystalline wax, synthetic wax, hydrogenated coco-glycerides, and a mixture thereof.

The body composition may further contain an oil. That is, the body composition may further contain an oily ingredient that is in a liquid phase at room temperature within the range where a solid phase is maintained. The liquid-phase oil may use any known oil that is acceptable as a cosmetic ingredient and, for example, the liquid-phase oil may include at least one selected from the group consisting of avocado oil, olive oil, almond oil, camellia oil, sesame oil, soybean oil, cottonseed oil, palm oil, jojoba oil, coconut oil, isopropyl palmitate, octyldodecyl myristate, cetyl ethylhexanoate, squalene, and a mixture thereof, but is not limited thereto.

The body composition may preferably contain an essential oil as a liquid-phase oil. In the present disclosure, the essential oil refers to an aromatic oil extracted from a plant.

The body composition of the present disclosure may further contain an additive that is commonly used in the art to which the present disclosure pertains, and for example, the additive may include at least one selected from the group consisting of a thickener, a surfactant, a colorant, pearl, a fragrance material, a preservative, a stabilizer, and a mixture thereof, but is not limited thereto.

### B. Core Composition

The core composition may use any cosmetic composition that may be used to fill a mold, but is not particularly limited thereto.

Hereinafter the core composition of the present disclosure will be illustratively described.

The cosmetic composition can be formulated as a solid, and in order to ensure the strength and stability of the formulation, the cosmetic composition may contain an ingredient that is commonly used in the art to which the present disclosure pertains, and may contain for example wax or a gelling agent.

The wax may be at least one selected from the group consisting of ceresin, ozokerite, paraffin wax, microcrystalline wax, carnauba wax, candelilla wax, Toxicodendron succedaneum fruit wax, lanolin, beeswax, and synthetic wax.

The gelling agent may be at least one selected from the group consisting of dimethicone/vinyl dimethicone crosspolymer, polyethers, polyamides, dibutyl lauroyl glutamide, dextrin palmitate, and bentonite.

The cosmetic composition may contain an oil, purified water, an emulsifier, and/or a powder according to the formulation and use of the cosmetic composition.

The oil may be at least one selected from the group consisting of hydrocarbon-based oils, ester-based oils, silicone-based oils, higher fatty acids, animal oils, and vegetable oils.

The emulsifier may be at least one selected from the group consisting of cationic emulsifiers, anionic emulsifiers, nonionic emulsifiers, natural emulsifiers, and silicone emulsifiers.

The powder may use an inorganic powder, an organic powder, or a combination thereof.

The cosmetic composition may further contain an additive that is commonly used in the art to which the present disclosure pertains, according to use. Examples of the additive may include at least one selected from the group consisting of a colorant, a thickener, a film forming agent, a surfactant, an ultraviolet blocker, a preservative, a moisturizer, a dispersant, a coloring, and a functional ingredient, but is not limited thereto.

The cosmetic composition may be applied to eyeshadows, eyebrow products, eyeliners, lip products, lip liners, concealers, blushers, and the like, but is not limited thereto.

Hereinafter, the method for manufacturing a cosmetic pencil of the present disclosure will be described by one example.

### C. Manufacturing of Cosmetic Pencil

A core composition was prepared by mixing respective ingredients as shown in the composition (wt %) in Table 1 below.

Specifically, weighing was conducted according to the respective ingredients and contents (wt %) thereof in Table 1 below. Thereafter, waxes and oils were weighed and placed in a mixing container, and then heated at 80 to 90° C. until they were completely melted into a transparent liquid. Subsequently, an inorganic pigment, an organic pigment, and a pearl pigment were added and then uniformly dispersed using an Agi-mixer at 2,500 rpm for 10 minutes.

TABLE 1

| | Content |
|---|---|
| Ingredient | (wt %) |
| C12-15 alkyl ethyl hexanoate | 15.93 |
| Glyceryl caprylate | 0.05 |
| Lecithin | 0.10 |
| VP/hexadecene copolymer | 2.00 |
| Vinyl dimethicone/methicone silsesquioxane crosspolymer | 0.10 |
| Isostearic acid | 0.10 |
| Isopropyl myristate | 0.10 |
| Ethylhexyl palmitate | 0.10 |
| Carnauba Wax | 5.00 |
| Caprylyl Glycol | 0.05 |
| Candelilla wax | 5.00 |
| Phenyl trimethicone | 12.00 |
| Polyglyceryl-2 triisostearate, | 10.00 |
| Polyglyceryl-3 Polyricinoleate | 0.07 |
| Polyisobutene | 2.00 |
| Polyhydroxystearic Acid | 0.40 |
| Synthetic wax | 10.00 |
| Inorganic pigment and organic pigment | 37.00 |
| Total | 100.00 |

Thereafter, the first mold shown in FIG. 3A was used to prepare core molded bodies.

Specifically, the core composition maintained at 80 to 90° C. was filled into the first mold maintained at 30 to 50° C., and then cooled at −10 to 30° C. for 5 to 10 minutes and then released, thereby manufacturing core molded bodies.

A body composition was prepared by mixing respective ingredients as shown in the composition (wt %) in Table 2 below.

Specifically, weighing was conducted according to the respective ingredients and contents (wt %) thereof in Table 2 below. Thereafter, waxes and oils were weighed and placed in a mixing container, and then heated at 80 to 90° C. until they were completely melted into a transparent liquid phase. Then, an inorganic pigment, an organic pigment, and a pearl pigment were added, and then uniformly dispersed using an Agi-mixer at 2,500 rpm for 10 minutes.

TABLE 2

| Ingredient | Content (wt %) |
|---|---|
| Microcrystalline wax | 35.00 |
| Hydrogenated soybean oil | 35.00 |
| Hydrogenated coco-glyceride | 15.11 |
| Synthetic wax | 12.00 |
| Inorganic pigment and organic pigment | 3.00 |
| Total | 100.00 |

Thereafter, as shown in FIG. 3B, the manufactured core molded bodies were inserted into the second mold, and then the body composition was injected, thereby molding bodies.

Specifically, the body composition maintained at 80 to 90° C. was filled into the first mold maintained at 30 to 50° C., and then cooled at −10 to 30° C. for 5 to 10 minutes and then released, thereby manufacturing bodies.

As such, a cosmetic pencil having a core disposed inside a body were manufactured.

### INDUSTRIAL APPLICABILITY

The present disclosure is applicable in cosmetics.

1. A cosmetic product comprising:
a body with a predetermined width and a predetermined length, wherein the body is elongated in the longitudinal direction and formed of a wax material; and
a core elongated in the longitudinal direction of the body, inside the body, and containing a cosmetic composition.

2. The cosmetic product of claim 1, comprising a lid coupled to either end of the body, wherein the lid contains at least one material selected from the group consisting of wax, oil, cellulose, gelatin, paper, felt paper, and leather.

3. The cosmetic product of claim 1, wherein the body has a length of 30 to 200 mm.

4. The cosmetic product of claim 1, wherein the core has a width of 2 to 20 mm.

5. The cosmetic product of claim 1, wherein the body further contains an oil.

6. The cosmetic product of claim 1, wherein the body further contains at least one selected from the group consisting of a colorant, pearl, and a fragrance material.

7. A cosmetic pencil comprising the body and the core of claim 1.

8. A method for manufacturing a cosmetic product, the method comprising:
a core molding step of filling a core composition into a first mold, followed by cooling and releasing, thereby preparing a core molded body; and
a body molding step of filling a body composition into the area outside the core molded body while the core body is inserted into the second mold, followed by cooling and releasing, thereby molding a body.

9. The method of claim 8, wherein in the body molding step, the body composition is filled to be equal to or higher than the height of the core molded body.
